(19) United States
(12) Patent Application Publication
(10) Pub. No.: US 2020/0306198 A1
(43) Pub. Date: Oct. 1, 2020

(54) METHOD FOR PRODUCING A PHARMACEUTICAL CARRIER

(71) Applicant: NOVARTIS AG, Basel (CH)

(72) Inventors: Florian Beck, Hallau (CH); Hans de Waard, Basel (CH); Sarah Gold, Frick (CH); Stefan Hirsch, Lorrach (DE); David Hook, Rheinfelden (CH); Nikhil Kavimandan, South Plainfield, NJ (US); Markus Krumme, Allschwil (CH); Steffen Lang, Reinach (CH); Detlef Moll, Hallau (CH); Siddharthya Mujumdar, Roschenz (CH); Anh-Thu Nguyen-Trung, Kembs (FR); Joerg Ogorka, Reinach (CH); Norbert Rasenack, Weil am Rhein (DE); Raphael Tobler, Hallau (CH); Patrick Tritschler, Freiburg (DE)

(21) Appl. No.: 16/652,107

(22) PCT Filed: Sep. 28, 2018
(86) PCT No.: PCT/IB2018/057540

§ 371 Date: Mar. 30, 2020

Related U.S. Application Data
(60) Provisional application No. 62/566,567, filed on Oct. 2, 2017.

(30) Foreign Application Priority Data
Feb. 1, 2018 (EP) 18154698.7

(51) Int. Cl. A61K 9/48

(52) U.S. Cl. CPC A61K 9/4833; A61K 9/4816; A61K 9/4866; A61K 9/485; A61K 9/4808

(57) ABSTRACT

A formulation for injection molding of a pharmaceutical carrier comprises 43.5-97% (w/w) of one or more polyethylene oxide polymer having a weight average molecular weight of M_{W} 94,000-188,000; and optionally one or more excipients.

The present invention relates to a formulation for injection molding of a pharmaceutical carrier used to enclose a pharmaceutical composition.

### BACKGROUND OF THE INVENTION

Two common dosage forms used to administer orally solid pharmaceutical compositions are filled hard capsules and compressed tablets. Hard capsules are typically made using gelatin. A common production method is to form the two parts by dipping stainless steel pins into a gelatin solution. The capsule halves are then stripped from the pins and trimmed before being joined to make each capsule. An alternative method of manufacture which can allow for more complex geometries is to use injection molding.

The composition of traditional capsules is limited to polymers which have suitable rheological and film forming properties when dispersed in water. Injection molding however, is a hot melt process, which necessitates very different material properties. This presents both an opportunity to move away from traditional capsule materials such as gelatin (animal derived, mechanical properties dependent on environmental conditions) and HPMC (dissolution lag time) and a challenge as the injection molding process is very demanding with respect to required material properties. The materials must be thermally stable during the process, have good melt flow properties—particularly under high shear conditions, be flexible enough when cooled to be ejected from the machine and for this application be mechanically strong to enable pharmaceutical processing and dissolve quickly in water. In addition the material must be suitable for human consumption and be approved for pharmaceutical use.

### SUMMARY OF THE INVENTION

We have developed a new formulation for injection molding of pharmaceutical carriers. In particular, the high performance of the formulation in the injection molding process enables flexibility in design of the carriers allowing for robust manufacture of design features with very small dimensions—traditionally a challenge in injection molding. The formulation allows preparing pharmaceutical carriers comprising very fine and thin design details by injection molding, which pharmaceutical carriers maintain stability and integrity following storage. At the same time, the formulation provides the pharmaceutical carrier with good dissolution rates.

In addition, we have developed a novel pharmaceutical dosage form, (also referred to herein as Prescido™) which is designed to have the functionality of a standard pharmaceutical capsule while maintaining the patient appeal of a tablet. The carriers described herein are manufactured via a precision injection molding process, using a formulation designed to perform well in thermal processes, such as the formulation of the invention. Design & manufacturing features together with their benefits include, inter alia, thin wall sections (fast carrier disintegration times in aqueous media), small snap close features (tight closure prevents opening of carrier during transport and limits tampering of carrier contents), numbering of cavities (traceability and sorting of parts before use) and high weight & dimension precision (robust handling processes).

Accordingly, the present invention is directed to a formulation for injection molding of a pharmaceutical carrier, wherein the formulation comprises 43.5-97% (w/w) of one or more polyethylene oxide polymer having a weight average molecular weight of M_{W} 94,000-188,000.

In one embodiment, the formulation further comprises 3-7% (w/w) of an anti-tackifier

The formulation may also comprise one or more excipients, as further defined below.

Hence, the formulation can be advantageously applied in a method for producing a pharmaceutical carrier comprising the steps of melting a formulation of the present disclosure and injecting the melt into a mold, and optionally cooling the injected melt and optionally ejecting the molded material. The method may comprise a further step of sorting the carrier parts by mold cavity. In preferred embodiments, the lid part and the bottom part are connected to each other by a complementary closing mechanism, in particular wherein the closing mechanism comprises a first snap part which projects from the bottom part so as to face and to interact with a second snap part which projects from the lid part. In further preferred embodiments, the bottom part of said pharmaceutical carrier is provided with an angled balcony adjacent to the first snap part, and which is inclined radially outwards, in particular from an outer circumference of the first snap part, which allows powder to fall out of the closing mechanism.

In addition to having excellent thermal processing properties, the formulation developed imparts a number of benefits to the carriers compared to traditional capsules, such as, for example, low water content (improved compatibility with water sensitive actives), low moisture absorption and sensitivity at standard manufacturing conditions, and very fast dissolution (rapid carrier rupture in aqueous media). Thus, carriers made using the formulation, such as the Prescido™ carriers described herein, have an advantage over traditional capsules due to having favorable water content & sorption properties, an advantage for processing and stability of water sensitive compounds.

Accordingly, in still another aspect, the present invention relates to a pharmaceutical carrier, produced by the method of the present disclosure using the formulation of the present disclosure, comprising a lid part and a bottom part wherein at least one of the lid part and the bottom part has a first wall section (26, 30) with a thickness of 180-220 μm, preferably 185-215 μm, even more preferably 190-210 μm, even more preferably 195-205 μm, and most preferably about 200 μm, and a second wall section with a thickness of 350-450 μm, preferably 375-425 μm, more preferably 390-410 μm, and most preferably about 400 μm. The first wall section of the lid part may define an entire top portion of the lid part. Alternatively or additionally thereto, the first wall section of the bottom part may define an entire bottom portion of the bottom part. In a particular preferred embodiment, the pharmaceutical carrier consists of the lid part and the bottom part, i.e. is designed in the form of a two-piece component without any additional elements. Further preferred embodiments are described herein below, and in the claims. One additional particular advantage is that the pharmaceutical carrier of the present disclosure exhibits a very low standard mass deviation of the respective carrier parts of less than 1 mg, preferably less than 0.8 mg, more preferably less than 0.6 mg, even more preferably less than 0.4 mg, still more preferably less than 0.3 mg, still even more preferably less than 0.2 mg, and most preferably less than 0.1 mg.

### BRIEF DESCRIPTION OF THE DRAWINGS

FIG. 1 shows various designs of a pharmaceutical carrier,

FIG. 2 shows sectional views of a lid part and a bottom part of an exemplary embodiment of the pharmaceutical carrier according to FIG. 1 including detailed views of a closing mechanism provided on the lid part and the bottom part,

FIG. 3A shows a three-dimensional view of the carrier bottom part as shown on the right in FIG. 2,

FIG. 3B shows a further detailed view of the closing mechanism provided on the lid part and the bottom part of the pharmaceutical carrier according to FIG. 2,

FIG. 4 shows a diagram indicating a dissolution profile of the pharmaceutical carrier depicted in FIGS. 1 to 3, and

FIG. 5 shows a diagram indicating an absolute mass variation of the pharmaceutical carrier depicted in FIGS. 1 to 3.

FIG. 6 shows photographs of pharmaceutical carriers prepared from polyethylene oxide having a weight average molecular weight of between 49,000 and 90,000, after storage at 25° C. and 60% room humidity. The figure shows the closure of flat pharmaceutical carriers (8 mm) after 3 months storage (A), and after six months storage (B). The carriers did not close well in (A), and got worse over time in (B), as more deformation is observed. Moreover, the figure shows the closure of slightly higher pharmaceutical carriers (12 mm) after 3 months of storage (C) and six months of storage (D). The photographs show a sticking to the mold mark in (C), and a worsening in the closure in (D).

FIG. 7 shows photographs of top and bottom parts of pharmaceutical carriers prepared from polyethylene oxide having a weight average molecular weight of between 49,000 and 90,000, after three months of storage at 30° C. and 75% room humidity (A), or 40° C. and 75% room humidity (B), (C). Several of the top and bottom parts show damage and/or deformation after three months storage.

### DETAILED DESCRIPTION OF THE INVENTION

Commercially available capsules are manufactured via a dip coating process. This involves having a reservoir of polymer/water mix and dipping in pins such that they become coated with the mix. The pins are then lifted out of the mix, and the polymer mix on the pin is dried to form a hard capsule before being removed. Prescido™ carriers on the other hand, are manufactured via injection molding. Injection molding involves melting of materials in a screw which is then used to inject the melt at high pressure into a mold where it is rapidly cooled before being ejected. This process has a number of advantages over dip coating: the process can be extremely precise, as electric drivers precisely control movement of the machine, which together with very tight control of process parameters such as temperature and pressure and precision mold manufacture, results in high uniformity of parts.

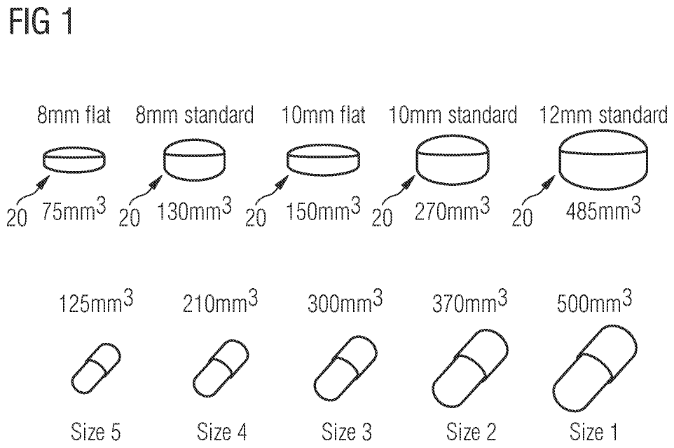

Prescido™ containers are capsules that are filled similar to a capsule formulation, but have appearance of a film-coated tablet. This creates additional presentation options for marketing to choose from in case a dosage form presentation other than a conventional capsule is desired. FIG. 1 (top row) shows a range of designs of the Prescido™ platform.

As becomes apparent from FIG. 1, the Prescido™ containers may have different designs and different filling volumes. Specifically, the containers may have various diameters and heights so that an appropriate container may be chosen, for example in dependence on the volume of powder to be filled into the containers.

In addition, the use of injection molding opens up opportunities for complicated part geometries. In dip molding, both the outer and inner geometries of the capsule are limited to the shape of the pins whereas the shape of injection molded parts is defined by the mold shape, which can allow multiple features on each face of the carrier.

The composition of traditional capsules is limited to polymers which have suitable rheological and film forming properties when dispersed in water. Injection molding however, is a hot melt process, which necessitates very different material properties. This presents both an opportunity to move away from traditional capsule materials such as gelatin (animal derived, mechanical properties dependent on environmental conditions) and HPMC (dissolution lag time) and a challenge as the injection molding process is very demanding with respect to required material properties. The materials must be thermally stable during the process, have good melt flow properties particularly under high shear conditions, be flexible enough when cooled to be ejected from the machine and for this application be mechanically strong to enable pharmaceutical processing and dissolve quickly in water. In addition the material must be suitable for human consumption and be approved for pharmaceutical use.

The present inventors have found that a formulation suitable for injection molding can be based on polyethylene oxide (PEO), see Example 1 herein below. Ratios of different molecular weight PEO were tested to achieve a formulation with the desired physico-chemical properties.

Accordingly, the present disclosure provides a formulation for injection molding of a pharmaceutical carrier, wherein the formulation comprises 43.5-97% (w/w) of one or more polyethylene oxide polymer having a weight average molecular weight of M_{W} 94,000-188,000.

As shown by Formulae 1-11 in the examples, suitable formulations for injection molding of a pharmaceutical carrier have a weight average molecular weight of M_{W} 94,000-188,000. In preferred embodiments, said polyethylene oxide polymer has a weight average molecular weight of M_{W} 95,000-185,500, more preferably of M_{W} 97,500-183,000, more preferably of M_{W} 100,000-175,000, more preferably of M_{W} 102,000-165,000, more preferably of M_{W} 105,000-150,000, even more preferably of 107,500-130,000, and most preferably of M_{W} 110,000-115,000.

As also reported in Example 1, it was found that pharmaceutical carriers prepared from molts having a weight average molecular weight of less than M_{W} 94,000 exhibited an undesirable integrity and/or stability. For example, pharmaceutical carriers stored at 25-40° C. and 60-75% room humidity did not close very well after 3 months or 6 months storage. Such problems did not occur when using molts having a weight average molecular weight of at least M_{W} 94,000. Moreover, pharmaceutical carriers made up of a polyethylene oxide polymer having a weight average molecular weight of more than M_{W} 188,000 resulted in pharmaceutical carriers in which the dissolution rate is unsatisfying. The relationship between an increase of the molecular weight polymer and a decrease in dissolution rate has been studied previously (see for example, Ueberreiter K. The solution process. In: Crank J, Park GS, editors. Diffusion in polymers. New York, N.Y.: Academic Press; 1968. p. 219-57; Miller-Chou, B and Koenig, J., A review of polymer dissolution, Prog. Polym. Sci. 2003, 28: 1223-1270).

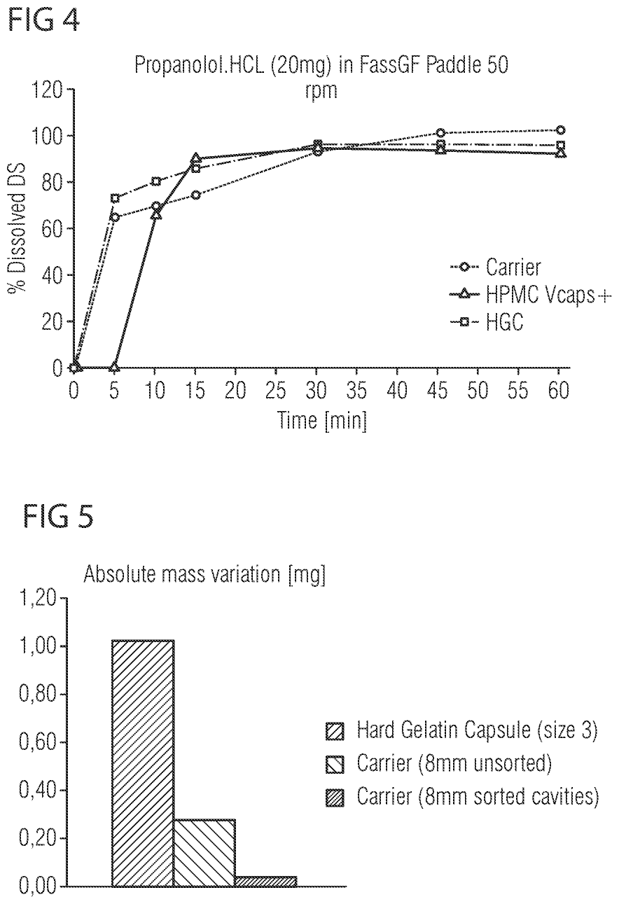

The dissolution rate of a capsule can be determined using the ‘assay for immediate release’ as described in the US Pharmacopeia, section <711> from 2011. The assay uses the USP apparatus II (paddle) and Fasted state simulating gastric fluid (FasSGF; commercially available) at 37° C. and 50 rpm with n=3 capsules. In one embodiment, the pharmaceutical carrier exhibits a dissolution rate of at least 30%, preferably at least 40%, more preferably at least 50%, more preferably at least 60%, more preferably at least 65%, and most preferably at least 70% drug substance within 5 minutes; e.g. using propanolol.HCl as the test substance. See also Example 2 and FIG. 4 herein.

As exemplified by Formula 1-11 in the examples, said polyethylene oxide polymer may comprise, preferably consist of, one or more polyethylene oxide having a weight average molecular weight of about M_{W} 100,000, polyethylene oxide having a weight average molecular weight of about M_{W} 200,000, polyethylene oxide having a weight average molecular weight of about M_{W} 300,000, polyethylene oxide having a weight average molecular weight of about M_{W} 600,000, and polyethylene oxide having a weight average molecular weight of M_{W} 8,000. Such polyethylene oxides are commercially available.

In a particular preferred embodiment, said polyethylene oxide polymer comprises 35-80% (w/w) of a first polyethylene oxide having a weight average molecular weight of M_{W} 100,000; and 4-28.5% (w/w) of a second polyethylene oxide having a weight average molecular weight of M_{W} 200,000. In further preferred embodiments, the formulation may comprise 41-77.5% (w/w), preferably 42-76% (w/w), more preferably 43-75% (w/w), more preferably 45-74% (w/w), more preferably 50-74% (w/w), and most preferably about 73.5% (w/w) of said first polyethylene oxide. In certain preferred embodiments the formulation comprises 4-27.5% (w/w), preferably 5-25% (w/w), more preferably 6-22% (w/w), more preferably 10-21% (w/w), more preferably 11-20.5% (w/w), and most preferably about 20% (w/w) of said second polyethylene oxide.

As further demonstrated in Example 1, the formulation may advantageously additionally comprise 3-7% (w/w) of an anti-tackifier. Specifically, it was found that the processibility of the PEO could be further improved in terms of faster cycle times, lower pressure and improved ejection and melt flow when adding 3-7% by weight of an anti-tackifier. In further embodiments, the formulation for injection molding of the pharmaceutical carrier comprises 3.5-6.5%, preferably 4-6% (w/w), even more preferably 4.5-5.5% (ww), and most preferably about 5% of the anti-tackifier. A particularly preferred anti-tackifier is talc.

The formulation may also comprises an excipient. The excipient may be at least one selected from the list consisting of colorant, antioxidant, opacifier, acid solubilizer, base solubilizer, surfactant, filler, glidant, lubricant, disintegrant, flavour, and sweetener. The acid solubilizer may be selected from the group consisting of tartaric acid, citric acid, fumaric acid, maleic acid, malic acid, and any combination thereof. The base solubilizer may be selected from the group consisting of tromethamine, sodium bicarbonate, sodium carbonate, and any combination thereof. The surfactant may be selected from the group consisting of sodium lauryl sulfate, poloxamer, docusate sodium, polyoxyethylene sorbitan fatty acid esters, polyoxyglycerides, polyoxyl hydrogenated castor oil. The filler may be selected from the group consisting of talc, mannitol, microcrystalline cellulose, dicalcium phosphate, calcium carbonate, magnesium aluminium metasilicate. The glidant may be colloidal silicon dioxide. The lubricant may be selected from the group consisting of stearic acid or one of its salts such as magnesium stearate or calcium stearate, hydrogenated vegetable oil, sodium stearyl fumarate (SSF), stearyl alcohol, glyceryl behenate, and any combination thereof. The disintegrant may be crospovidone, sodium starch glycolate, sodium croscarmellose, or any combination thereof. The opacifier and colorant may be selected from titanium dioxide, iron oxide, lake pigments, mica-based pigments (e.g., Candurin), formulated pigments (e.g., Opadry®), and any combination thereof. The antioxidant may be selected from the group consisting of butylated hydroxy toluene, butylated hydroxy anisole, vitamin E, vitamin E TPGS, ascorbic acid, isoascorbic acid, citric acid, propyl gallate, and any combination thereof. Further excipients and examples of colorant, antioxidant, opacifier, acid solubilizer, base solubilizer, surfactant, filler, glidant, lubricant, disintegrant, flavour, and sweetener will be apparent to the skilled person, and described in well-known reference books such as Remington, Handbook of Pharmaceutical Excipients.

In one embodiment, the formulation comprises 0-6% (w/w) of one or more colorant and/or opacifier, preferably 0.01-5% (w/w) of one or more colorant and/or opacifier, more preferably 0.25-4% (w/w) of one or more colorant and/or opacifier, more preferably 0.5-3% (w/w) of one or more colorant and/or opacifier, more preferably 0.75-2.5% (w/w) of one or more colorant and/or opacifier, more preferably 1-2% (w/w) of one or more colorant and/or opacifier, more preferably 1-1.5% (w/w) of one or more colorant and/or opacifier, and most preferably about 1% (w/w) of one or more colorant and/or opacifier.

It is further preferred that the formulation comprises 0.01-1% (w/w) of an antioxidant, preferably 0.05-0.8% (w/w) of an antioxidant, more preferably 0.1-0.75 (w/w) of an antioxidant, more preferably 0.2-0.7 (w/w) of an antioxidant, more preferably 0.3-0.6 (w/w) of an antioxidant, more preferably 0.4-0.5 (w/w) of an antioxidant, and most preferably about 0.5% (w/w) of an antioxidant.

In certain embodiments, the formulation comprises 30-38% (w/w) of a filler, preferably 32-38% (w/w), more preferably 34-36% (w/w); in particular wherein the filler is talc.

One particularly suitable embodiment of the formulation is shown in Formula 11 in the examples. In this embodiment, the formulation comprises 73.5% (w/w) of a first polyethylene oxide having a weight average molecular weight of M_{w} 100,000, 20% (w/w) of a second polyethylene oxide having a weight average molecular weight of M_{w} 200,000, 5% (w/w) talc, 1% (w/w) colorant and/or opacifier, and 0.5% (w/w) of an antioxidant.

The present disclosure further provides a method of producing a pharmaceutical carrier, comprising the steps of (a) melting a formulation as described above, and (b) injecting the melt into a mold. Said method may optionally comprise a further step (c) cooling the injected melt and optionally ejecting the molded material. As described above, preferably the pharmaceutical carrier is a capsule, and at least one lid part and at least one bottom part is formed.

At least one of the lid part and the bottom part has a first wall section with a thickness of 180-250 μm, preferably 185-225 μm, and even more preferably 190-220 μm, and a second wall section with a thickness of 350-450 μm, preferably 375-425 μm, more preferably 390-410 μm, and most preferably about 400 μm. In a preferred embodiment the first wall section (26) of the lid part (22) defines an entire top portion of the lid part (22) and/or wherein the first wall section (30) of the bottom part (24) defines an entire bottom portion of the bottom part (24).

In a particular preferred embodiment, the pharmaceutical carrier consists of the lid part and the bottom part, i.e. is designed in the form of a two-piece component without any additional elements.

Preferably, the pharmaceutical carrier is tablet shaped, i.e. designed to have the functionality of a standard pharmaceutical capsule while maintaining the patient appeal of a tablet. In particular, the containers are typically selected to have a tablet shape, such as a disc shape, as opposed to a capsule shape. When considering the lid and bottom part of the pharmaceutical carrier, a capsule shape would be elongated along a central axis running from a center of the bottom part to a center of the lid part. Thus for a traditional capsule, a ratio of a lateral extension, in particular a diameter of the lid and bottom part to a height of the assembled lid and bottom parts along the central axis would be less than 1:1, such as 0.5:1 or less. For example a type 000 capsule has a diameter of 5.32 mm and a height of 14.3 mm (ratio of 0.37:1) and a type 4 capsule has a diameter of 9.55 mm and a height of 26.1 mm (also a ratio of 0.37:1). By contrast a tablet-shaped carrier has a flatter shape and would have a ratio of greater than 1 (1:1 being essentially a sphere). Thus, the pharmaceutical carrier preferably is designed such that the ratio of a lateral extension, in particular a diameter of the lid and bottom part to the height of the assembled lid and bottom parts is >1, preferably ≥1.4, more preferably ≥1.5, even more preferably ≥2, most preferably ≥2.4 and in particular ≥2.5. The containers depicted in FIG. 1, from left to right, have a ratio of a lateral extension of the lid part and the bottom part to a height of the assembled lid and bottom parts of 1:0.4, i.e. of 2.5, 1:0.7, i.e. of 1.43, 1:0.42, i.e. of 2.38, 1:0,875, i.e. 1.14, 1:0,69, i.e. of 1.45.

The thickness of the first wall section has been optimized at 190 to 220 μm. This is thick enough such that, during manufacturing of the pharmaceutical carrier via injection molding, the material can flow through the thin first wall section, and still reliably fill the thicker walled area of the second wall section while being thin enough to achieve the rapid carrier disintegration required to achieve immediate release dissolution profiles of filled compounds. The second wall section has been optimized to a thickness of 400 μm. Here the balance is between having a greater internal volume available for filling, and having the mechanical strength required for filling and handling (including resistance to opening once filled).

A first wall section of the lid part may define at least a portion of a top portion of the lid part. Preferably, the first wall section of the lid part defines the entire top portion of the lid part such that, upon disintegration of the thin first wall section, a rapid and reliable release of compounds filled into the pharmaceutical carrier via the disintegrating top portion of the lid part is achieved.

A second wall section of the lid part may define at least a portion of a side wall portion of the lid part. For example, the second wall section of the lid part may define a shoulder or corner of the lid part which is arranged adjacent to the top portion of the lid part. Specifically, the second wall section of the lid part may extend from the first wall section, i.e. in particular the top portion of the lid part, along an outer circumference thereof, in the direction of the bottom part. This design provides the lid part with the mechanical stability which is required to handle the lid part and to connect it with the bottom part so as to form the pharmaceutical carrier as desired.

In the context of this application, the expression “side wall portion of the lid part” defines a portion of the lid part which extends substantially parallel to the central axis of the pharmaceutical carrier. Preferably, the side wall portion of the lid part has a circular cylindrical shape and surrounds the central axis of the pharmaceutical carrier substantially parallel therewith. The expression “top portion of the lid part” defines a portion of the lid part which is connected to the side wall portion and “covers” a free space surrounded by side wall portion at one end thereof. The top portion of the lid part might extend substantially perpendicular with respect to the central axis of the pharmaceutical carrier, wherein, however, in a particular preferred embodiment, the top portion is at least slightly curved with respect to the central axis of the pharmaceutical carrier. When viewed from “outside” of the carrier, the top portion of the lid part in particular is provided with a concave curvature.

In a preferred embodiment of the pharmaceutical carrier, a first wall section of the bottom part defines at least a portion of a bottom portion of the bottom part. Preferably, the first wall section of the bottom part defines the entire bottom portion of the bottom part such that, upon disintegration of the thin first wall section, a rapid and reliable release of compounds filled into the pharmaceutical carrier via the disintegrating bottom portion of the bottom part is achieved.

A second wall section of the bottom part may define at least a portion of a side wall portion of the bottom part. Specifically, the second wall section of the bottom part may extend from the first wall section, i.e. in particular the bottom portion of the bottom part, along an outer circumference thereof, in the direction of the lid part. Preferably, the height of the second wall section of the bottom part is larger than the height of the second wall section of the lid part. In other words, in a preferred embodiment of the pharmaceutical carrier, the bottom part has a generally hollow cylindrical shape and hence defines a “vessel” which may be filled with the pharmaceutical compound. To the contrary, the lid part, which may be provided with a second wall section which merely defines a shoulder or corner surrounding the top portion of the lid part, may have a generally “flat” shape. The larger wall thickness of the second wall section as compared to the first wall section provides the bottom part with a mechanical strength and stability which allows an unhindered filling of the bottom part with the pharmaceutical compound.

In the context of this application, the expression “side wall portion of the bottom part” defines a portion of the bottom part which extends substantially parallel to the central axis of the pharmaceutical carrier. Preferably, the side wall portion of the bottom part has a circular cylindrical shape and surrounds the central axis of the pharmaceutical carrier substantially parallel therewith. The expression “bottom portion of the bottom part” defines a portion of the bottom part which is connected to the side wall portion and “covers” a free space surrounded by side wall portion at one end thereof. The bottom portion of the lid part might extend substantially perpendicular with respect to the central axis of the pharmaceutical carrier, wherein, however, in a particular preferred embodiment, the bottom portion is at least slightly curved with respect to the central axis of the pharmaceutical carrier. When viewed from “outside” of the carrier, the bottom portion of the bottom part in particular is provided with a concave curvature.

In preferred embodiments, the lid part and the bottom part are connected to each other by a complementary closing mechanism. The complementary closing mechanism provides for a reliable and easy to establish connection between the lid part and the bottom part.

More specifically, the closing mechanism may comprise a first snap part which projects from the second wall section of the bottom part so as to face and to interact with a second snap part which projects from the second wall section of the lid part. Upon closing the pharmaceutical carrier, i.e. upon connecting the lid part to the bottom part, at least one of the first and the second snap part may be elastically deformed. When the lid part and the bottom part have reached their final relative positions, i.e. when the lid part is positioned on top of the bottom part so as to seal the interior of the bottom part as desired, the elastic information of the at least one of the first and the second snap part may be released in such a manner that the snap parts intact with each other so as to reliably connect the lid part and the bottom part.

For example, the first snap part may comprise a projection which is adapted to engage with a corresponding projection provided on the second snap part so as to counteract separation of the first snap part and the second snap part and thus separation of the lid part and the bottom part. In particular, the projection of the first snap part may comprise a first abutting surface which faces the bottom part and which is adapted to abut against a second abutting surface which is formed on the second snap part and which faces the lid part when the bottom part and the lid part are connected to each other. The first abutting surface formed on the first snap part may extend at an angle of 90 to 150° relative to the side wall portion of the bottom part. The second abutting surface formed on the second snap part may extend at an angle of 90 to 150° relative to the side wall portion of the lid part.

The projection provided on the first snap part may taper in a direction of a free end of the first snap part so as to form a first inclined engagement surface. The first inclined engagement surface may be adapted to engage with a second inclined engagement surface formed on the projection provided on the second snap part which tapers in a direction of a free end of the second snap part. Upon connecting the lid part to the bottom part of the pharmaceutical carrier, the second inclined engagement surface may slide along the first inclined engagement surface thus guiding the projection provided on the first snap part into engagement with the corresponding projection provided on the second snap part. As a result, connecting the lid part to the bottom part is simplified.

One of the first and the second snap part may project from the second wall section of the lid part or the bottom part in the region of an inner circumference of the second wall section, wherein the other one of the first and the second snap part may project from the second wall section of the lid part or the bottom part in the region of an outer circumference of the second wall section of the bottom part. Preferably, the first snap part provided on the bottom part of the pharmaceutical carrier extends from the second wall section of the bottom part in the region of an inner circumference of the second wall section. A thus designed first snap part is particularly suitable for interaction with a second snap part which projects from a particularly shoulder- or corner-shaped second wall section of the lid part in the region of an outer circumference of the second wall section of the lid part.

The closing mechanism may further comprise an inner rib which projects from the second wall section of the lid part or the bottom part in the region of an inner circumference of the second wall section at a distance from the first or the second snap part which projects from the second wall section of the lid part or the bottom part in the region of an outer circumference of the second wall section. In particular, the closing mechanism may comprise inner rib which projects from the second wall section of the lid part in the region of an inner circumference thereof and hence at a distance from the second snap part which projects from the particularly shoulder- or corner-shaped second wall section of the lid part in the region of an outer circumference thereof. As a result, the inner rib and the second snap part define a gap therebetween which is adapted to accommodate the first snap part when the lid part and the bottom part of the pharmaceutical carrier are connected to each other. In the connected state of the lid part and the bottom part, the first snap part is held in place in the gap between the inner rib and the second snap part due to the interaction with the second snap part, i.e. in particular you to the interaction of the first abutting surface formed on the first snap part with the second abutting surface formed on the second snap part, while the inner rib provides for additional mechanical stability and stiffness of the closing mechanism.

It is, however, also conceivable to provide the bottom part of the pharmaceutical carrier with an inner rib, in particular in case the bottom part is provided with a first snap part which projects from the second wall section of the bottom part in the region of an outer circumference thereof and which is adapted to interact with a second snap part which projects from the second wall section of the lid part in the region of an inner circumference thereof. In this case, the inner rib and the first snap part may define a gap therebetween which is adapted to accommodate the second snap part when the lid part and the bottom part of the pharmaceutical carrier are connected to each other.

Preferably, the inner rib is shorter than the snap part arranged opposite to the inner rib. In other words, preferably, the snap part which, together with the inner rib, defines a gap for accommodating the other snap part projects further from the second wall section of the lid part or the bottom part than the inner rib. Further, the inner rib may taper in a direction of a free end of the inner rib so as to form a third inclined engagement surface facing the first or the second snap part which projects from the second wall section of the lid part or the bottom part in the region of an outer circumference of the second wall section and hence is arranged opposite to the inner rib. Preferably, the third inclined engagement surface provided on the inner rib extends substantially parallel to the abutting surface provided on the projection of the snap part arranged opposite to the inner rib. As a result, the snap part which is adapted to be accommodated in the gap defined between the inner rib and the snap part arranged opposite to the inner rib upon connecting the lid part and the bottom part of the pharmaceutical carrier is guided into engagement with the snap part arranged opposite to the inner rib.

In a preferred embodiment of the pharmaceutical carrier, the first wall section of the lid part, in particular in a region which is defined by a material injection point into a mold upon manufacturing of the lid part, is provided with a depression. This depression may have a wall thickness that is larger than the wall thickness of the remaining part of the first wall section, but smaller than the wall thickness of the second wall section of the lid part. For example, the depression may be arranged in a central region of a top portion of the lid part. A sign which indicates a cavity in which the lid part was molded on a multicavity molding tool during an injection molding process may be imprinted onto a surface, in particular an inner surface of the depression. This allows for automatic sorting of the lid parts by cavity for applications where tight weight uniformity is required.

Alternatively or additionally thereto, the first wall section of the bottom part, in particular in a region which is defined by a material injection point into a mold upon manufacturing of the bottom part, is provided with a depression. This depression may have a wall thickness that is larger than the wall thickness of the remaining part of the first wall section, but smaller than the wall thickness of the second wall section of the lid part. For example, the depression may be arranged in a central region of a bottom portion of the bottom part. A sign which indicates a cavity in which the bottom part was molded on a multicavity molding tool during an injection molding process may be imprinted onto a surface, in particular an inner surface of the depression. This allows for automatic sorting of the bottom parts by cavity for applications where tight weight uniformity is required.

At least one of the lid part and the bottom part, in the region of an inner surface thereof, may be provided with a plurality of inner protrusions which project radially inwards from an inner surface of the second wall section and/or an inner surface of the inner rib. In case the lid part or the bottom part which is provided with inner protrusions also is provided with an inner rib, the inner protrusions, in a direction of a central axis of the lid part or the bottom part, may extend from the top portion of the lid part or the bottom portion of the bottom part along the second wall section of the lid part of the bottom part and finally along the inner rib which projects from the second wall section in the region of an inner circumference thereof. In case the lid part of the bottom part which is provided with inner protrusions does not comprise an inner rib, the inner protrusions, in a direction of a central axis of the lid part or the bottom part, may extend from the top portion of the lid part or the bottom portion of the bottom part along the second wall section of the lid part or the bottom part. At least one of and in particular each of the inner protrusions may comprise a projecting nose which projects beyond the second wall section and/or the inner rib.

The inner protrusions, in particular when being provided with projecting noses, reduce a phenomenon termed ‘nesting’, i.e. an adherence of the parts and/or bottom parts stacked on top of each other. As a result, difficulties during manual and automated handling which may be caused by ‘nests’ of stacked parts which are difficult to separate can be eliminated.

In a preferred embodiment of the pharmaceutical carrier, the bottom part is provided with an angled balcony. The angled balcony may be formed in the region of an outer surface of the second wall section of the bottom part, in particular adjacent to the first snap part. The angled balcony may be inclined radially outwards from an outer circumference of the first snap part towards an outer surface of the second wall section. Powdery compounds to be filled into the pharmaceutical carrier which inadvertently fall onto the balcony of the bottom part upon filling or closing the pharmaceutical carrier can easily be removed.

The pharmaceutical carrier may be filled with neat API. In this context, the expression “neat API” designates an API comprising at most 5% (w/w) of an additive throughout all development stages of the pharmaceutical drug including its final commercial production. In particular, the neat API within the pharmaceutical carrier may comprise at most 5% (w/w) of an additive, preferably at most 4% (w/w), more preferably at most 3% (w/w), even more preferably at most 2% (w/w), and most preferably at most 1% (w/w).

In a preferred embodiment, a pharmaceutical carrier comprises a lid part and a bottom part. At least one of the lid part and the bottom part has a first wall section with a thickness of 180-250 μm, preferably 185-225 μm, and even more preferably 190-220 μm, and a second wall section with a thickness of 350-450 μm, preferably 375-425 μm, more preferably 390-410 μm, and most preferably about 400 μm. The first wall section of the lid part defines an entire top portion of the lid part. Alternatively or additionally thereto, the first wall section of the bottom part defines an entire bottom portion of the bottom part.

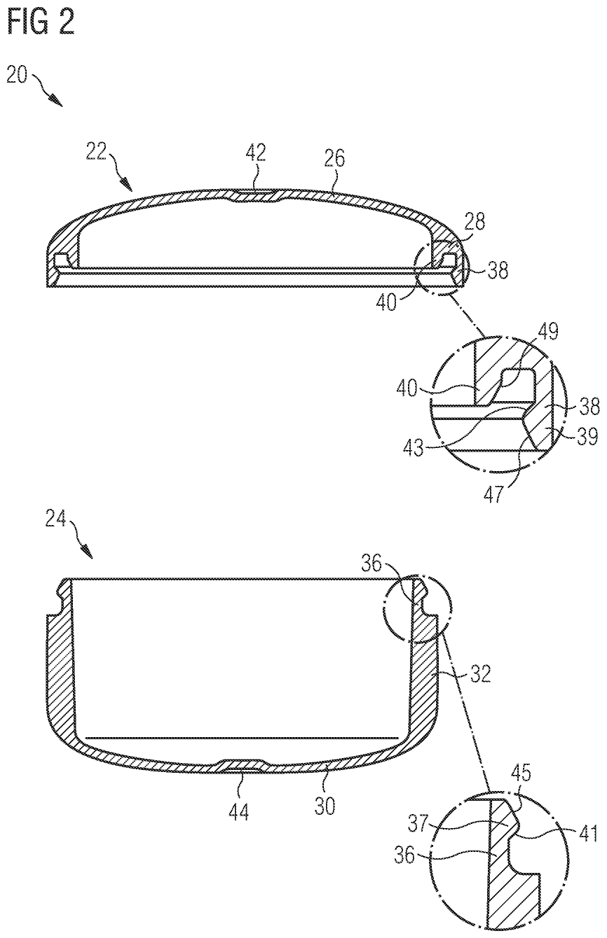

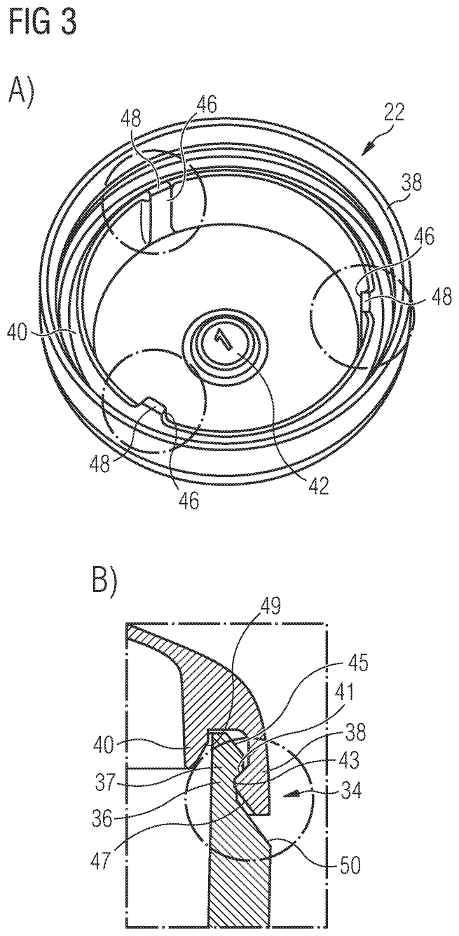

An exemplary pharmaceutical carrier 20 as depicted in FIG. 1 is shown in greater detail in FIGS. 2, 3A and 3B. The carrier 20 comprises a lid part 22 and a bottom part 24.

Specifically, the carrier 20 is designed of a two-part component and consists of the lid part 22 and the bottom part 24. The lid part 22, which is shown on the left in FIG. 2 and in FIG. 3A, comprises a first wall section 26 which defines a top portion of the lid part 22 and a second wall section 28 which defines a side wall portion of the lid part 22. In particular, the second wall section 28 of the lid part 22 defines a shoulder or corner of the lid part 22 which is arranged adjacent to the top portion of the lid part 22. Specifically, the second wall section 28 of the lid part 22 extends from the top portion of the lid part 22, along an outer circumference thereof, in the direction of the bottom part 24. The first wall section 26 has a wall thickness that is smaller than a wall thickness of the second wall section 28. In the preferred embodiment of the carrier 20 shown in FIG. 2, the first wall section 26 has a wall thickness of 190 to 220 μm, whereas the second wall section 28 has a wall thickness of about 400 μm.

Similarly, the bottom part 24, which is shown on the right in FIG. 2, comprises a first wall section 30 which defines a bottom portion of the bottom part 24 and a second wall section 32 which defines a side wall portion of the bottom part 24. The second wall section 32 of the bottom part 24 extends from the bottom portion of the bottom part 24 along an outer circumference thereof in the direction of the lid part 22. The first wall section 30 has a wall thickness that is smaller than a wall thickness of the second wall section 32. In the preferred embodiment of the carrier 20 shown in FIG. 2, the first wall section 30 has a wall thickness of 190 to 220 μm, whereas the second wall section 32 has a wall thickness of about 400 μm.

The lid part 22 and the bottom part 24 are connected to each other by means of a complementary closing mechanism 34 which is illustrated in greater detail in the detailed views shown in FIG. 2 as well as in FIG. 3B. The closing mechanism 34 comprises a first hook-shaped snap part 36 which projects from the second wall section 32 of the bottom part 24 in the region of an inner circumference of the second wall section 32. The first hook-shaped snap part 36 faces and interacts with a correspondingly shaped second hook-shaped snap part 38 which projects from the second wall section 28 of the lid part 22 in the region of an outer circumference of the second wall section 28. It would, however, also be conceivable to provide the closing mechanism 34 with a first snap part 36 which projects from the second wall section 32 of the bottom part 24 in the region of an outer circumference of the second wall section 32 and a second snap part 36 which projects from the second wall section 28 of the lid part 22 in the region of an inner circumference of the second wall section 28.

As becomes apparent from the detailed views shown in FIG. 2 and FIG. 3B, the first snap part 36 comprises a projection 37 which, upon connecting the lid part 22 and the bottom part 24, is adapted to engage with a corresponding projection 39 provided on the second snap part 38. The projection 37 of the first snap part 36 comprises a first abutting surface 41 which faces the bottom part 24. Similarly, the projection 39 of the lid part 22 comprises a second abutting surface 43 which faces the lid part 22. The first abutting surface 41 formed on the projection 37 of the first snap part 36 extends at an angle of approximately 135° relative to the side wall portion of the bottom part 24. The second abutting surface 43 formed on the projection 39 of the second snap part 38 extends at an angle of approximately 135° relative to the side wall portion of the lid part 22. Further, the projection 37 provided on the first snap part 36 tapers in a direction of a free end of the first snap part 36 so as to form a first inclined engagement surface 45. Similarly, the projection 39 provided on the second snap part 38 also tapers in a direction of a free end of the first snap part 38 so as to form a second inclined engagement surface 47.

The closing mechanism 34 further comprises an inner rib 40 which projects from the shoulder- or corner-shaped second wall section 28 of the lid part 22 in the region of an inner circumference of the second wall section 28. Hence, the inner rib 40 projects from the second wall section 28 of the lid part 22 at a distance from the second snap part 36 which projects from the second wall section 28 of the lid part 22 in the region of an outer circumference of the second wall section 28. As a result, the inner rib 40 and the second snap part 38 define a gap therebetween which is adapted to accommodate the first snap part 36 when the lid part 22 and the bottom part 24 of the pharmaceutical carrier 20 are connected to each other. However, in case the lid part 22 is provided with a second snap part 38 which is arranged in the region of an inner circumference of the second wall section 28 so as to interact with a first snap part 38 which is arranged in the region of outer circumference of the second wall section 32 of the bottom part 24, it is also conceivable that the closing mechanism 34 comprises an inner rib 40 which projects from the second wall section 32 of the bottom part 24 in the region of an inner circumference of the second wall section 32. In this case it is the first snap part 36 which, together with the inner rib 40, defines a gap which is adapted to accommodate the second snap part 38 when the lid part 22 and the bottom part 24 of the pharmaceutical carrier 20 are connected to each other.

The inner rib 40 is shorter than the second snap part 38 arranged opposite to the inner rib 40, i.e. the second snap part 38 projects further from the second wall section 28 of the lid part 22 than the inner rib 40. Further, the inner rib 40 tapers in a direction of a free end of the inner rib 40 so as to form a third inclined engagement surface 49 facing the second snap part 38 which projects from the second wall section 28 of the lid part 22 in the region of an outer circumference of the second wall section 28 and opposite to the inner rib 40. The third inclined engagement surface 49 extends substantially parallel to the second abutting surface 43 provided on the projection 39 of the second snap part 38 arranged opposite to the inner rib 40. In case the lid part 22 is provided with a second snap part 38 which is arranged in the region of an inner circumference of the second wall section 28 so as to interact with a first snap part 38 which is arranged in the region of outer circumference of the second wall section 32 of the bottom part 24, the third inclined engagement surface 49 formed on the inner rib 40 may face the first snap part 36 which projects from the second wall section 32 of the bottom part 24 in the region of an outer circumference of the second wall section 32 and opposite to the inner rib 40

Upon closing the pharmaceutical carrier 20, i.e. upon connecting the lid part 22 to the bottom part 24, the first inclined engagement surface 45 provided on the projection 37 of the first snap part 36 comes into contact with the second inclined engagement surface 47 provided on the projection 39 of the second snap part 38. When the lid part 22 approaches the bottom part 24, the second inclined engagement surface 47 slides along the first inclined engagement surface 45 which results in a slight elastic deformation of the first and the second snap part 36, 38. Specifically, the first snap part 38 is slightly bent radially inwards, whereas the second snap part 36 is slightly bent radially outwards. Inward bending of the first snap part 38 is, however, limited by the inner rib 40. Further, the third inclined engagement surface 49 provided on the inner rib 40 guides the second snap part 38 into its final position in the gap defined between the second snap part 38 and the inner rib 40, see FIG. 3B.

When the lid part 22 and the bottom part 24 have reached their final relative positions, i.e. when the lid part 22 is positioned on top of the bottom part 24 so as to seal the interior of the bottom part 24, the elastic deformation of the first and the second snap part 36, 38 is released and the first abutting surface 41 provided on the projection 37 of the first snap part 36 abuts against the second abutting surface 43 provided on the projection 39 of the second snap part 38. The interaction between the first and the second abutting surface 41, 43 contacts separation of the bottom part 24 and the lid part 22. The inner rib 40 provides for additional mechanical stability and stiffness of the closing mechanism 34.

The first wall section 26 of the lid part 22, in a central region which is defined by a material injection point into a mold upon manufacturing of the lid part 22, is provided with a depression 42 which has a wall thickness that is larger than the wall thickness of the remaining part of the first wall section 26, but still smaller than the wall thickness of the second wall section 28 of the lid part 22. A number, in the drawings the number “1”, is imprinted onto an inner surface of the depression 42 which indicates a cavity in which the lid part 22 was molded on a multicavity molding tool. Similarly, also the first wall section 30 of the bottom part 24, in a central region which is defined by a material injection point into a mold upon manufacturing of the bottom part 24, is provided with a depression 44 which has a wall thickness that is larger than the wall thickness of the remaining part of the first wall section 30, but still smaller than the wall thickness of the second wall section 32 of the bottom part 24. A number (not shown in the drawings) is imprinted onto an inner surface of the depression 44 which indicates a cavity in which the bottom part 24 was molded on a multicavity molding tool.

As becomes apparent from FIG. 3A, the lid part 22 further is provided with a plurality of inner protrusions 46 which project radially inwards from an inner surface of the second wall section 28 and an inner surface of the inner ring 40, respectively. In the specific embodiment of a lid part 22 shown in the drawings, three inner protrusions 46 are provided. It is, however, also conceivable to provide the lid part 22 with less than or more than three inner protrusions 46. The inner protrusions 46 serve to prevent jamming of parts 22, which are stacked on top of each other during handling. Each of the inner protrusions 46 comprises a nose 48 which projects from the inner rib 40 and which further reduces the risk of jamming of parts 22 stacked on top of each other. In the embodiment of the carrier 20 which is illustrated in the drawings, only the lid part 22 is provided with inner protrusions 46. It is, however, also conceivable that alternatively or additionally also the bottom part 24 of the carrier 20 is provided with inner protrusions as described herein.

Finally, as becomes apparent from FIG. 3B, the bottom part 24 is provided with an angled balcony 50 which is formed in the region of an outer surface of the second wall section 32 adjacent to the first hook-shaped snap part 36 and which is inclined radially outwards from an outer circumference of the hook-shaped snap part 38 towards an outer surface of second wall section 32. Powder which inadvertently falls onto the balcony 50 upon closing the carrier 20 can easily be removed.

Advantageously, the pharmaceutical carrier exhibits a standard mass deviation of the respective carrier parts of less than 1 mg, preferably less than 0.8 mg, more preferably less than 0.6 mg, even more preferably less than 0.4 mg, still more preferably less than 0.3 mg, still even more preferably less than 0.2 mg, and most preferably less than 0.1 mg, as shown in the examples section herein below.

The invention is further described by the following embodiments.
1. A formulation for injection molding of a pharmaceutical carrier, wherein the formulation comprises 43.5-97% (w/w) of one or more polyethylene oxide polymer having a weight average molecular weight of M_{W} 94,000-188,000; and optionally one or more excipients.
2. The formulation of embodiment 1, wherein said polyethylene oxide polymer comprises one or more polyethylene oxide having a weight average molecular weight of about M_{W} 100,000, polyethylene oxide having a weight average molecular weight of about M_{W} 200,000, polyethylene oxide having a weight average molecular weight of about M_{W} 300,000, polyethylene oxide having a weight average molecular weight of about M_{W} 600,000, and polyethylene oxide having a weight average molecular weight of M_{W} 8,000.
3. The formulation of embodiment 1 or 2, wherein said polyethylene oxide polymer comprises 35-80% (w/w) of a first polyethylene oxide having a weight average molecular weight of M_{W} 100,000; and 4-28.5% (w/w) of a second polyethylene oxide having a weight average molecular weight of M_{W} 200,000.
4. The formulation of embodiment 3, wherein the formulation comprises 41-77.5% (w/w), preferably 42-76% (w/w), more preferably 43-75% (w/w), more preferably 45-74% (w/w), more preferably 50-74% (w/w), and most preferably about 73.5% (w/w) of said first polyethylene oxide.
5. The formulation of any one of embodiments 3 or 4, wherein the formulation comprises 4-27.5% (w/w), preferably 5-25% (w/w), more preferably 6-22% (w/w), more preferably 10-21% (w/w), more preferably 11-20.5% (w/w), and most preferably about 20% (w/w) of said second polyethylene oxide.
6. The formulation of any one of embodiments 1-5, wherein the formulation comprises 3-7% (w/w) of an anti-tackifier, preferably 3.5-6.5% (w/w), preferably 4-6% (w/w), even more preferably 4.5-5.5% (w/w), and most preferably about 5% of the anti-tackifier.
7. The formulation of embodiment 6, wherein the anti-tackifier is talc.
8. The formulation of any one of embodiments 1-7, wherein the excipient is at least one selected from the list consisting of colorant, antioxidant, opacifier, acid solubilizer, base solubilizer, surfactant, filler, glidant, lubricant, disintegrant, flavour, sweetener.
9. The formulation of embodiment 8, wherein the acid solubilizer is selected from the group consisting of tartaric acid, citric acid, fumaric acid, maleic acid, malic acid, and any combination thereof.
10. The formulation of embodiment 8, wherein the base solubilizer is selected from the group consisting of tromethamine, sodium bicarbonate, sodium carbonate, and any combination thereof.
11. The formulation of embodiment 8, wherein the surfactant is selected from the group consisting of sodium lauryl sulfate, poloxamer, docusate sodium, polyoxyethylene sorbitan fatty acid esters, polyoxyglycerides, polyoxyl hydrogenated castor oil, and any combination thereof.
12. The formulation of embodiment 8, wherein the filler is selected from the group consisting of talc, mannitol, microcrystalline cellulose, dicalcium phosphate, calcium carbonate, magnesium aluminium metasilicate, and any combination thereof.
13. The formulation of embodiment 8, wherein the glidant is colloidal silicon dioxide, and/or wherein the lubricant is stearic acid or one of its salts such as magnesium stearate or calcium stearate, hydrogenated vegetable oil, sodium stearyl fumarate (SSF), stearyl alcohol, and/or glyceryl behenate, or any combination thereof.
14. The formulation of embodiment 8, wherein the disintegrant is crospovidone, sodium starch glycolate, sodium croscarmellose, or any combination thereof.
15. The formulation of embodiment 8, wherein the opacifier and colorant is selected from titanium dioxide, iron oxide, lake pigments, mica-based pigments, formulated pigments, and any combination thereof. 16. The formulation of embodiment 8, wherein the antioxidant is selected from the group consisting of butylated hydroxy toluene, butylated hydroxy anisole, vitamin E, vitamin E TPGS, ascorbic acid, isoascorbic acid, citric acid, propyl gallate, and any combination thereof.
17. The formulation of any one of embodiments 1-16, comprising 0-6% (w/w) of one or more colorant and/or opacifier, preferably 0.01-5% (w/w) of one or more colorant and/or opacifier, more preferably 0.25-4% (w/w) of one or more colorant and/or opacifier, more preferably 0.5-3% (w/w) of one or more colorant and/or opacifier, more preferably 0.75-2.5% (w/w) of one or more colorant and/or opacifier, more preferably 1-2% (w/w) of one or more colorant and/or opacifier, more preferably 1-1.5% (w/w) of one or more colorant and/or opacifier, and most preferably about 1% (w/w) of one or more colorant and/or opacifier.
18. The formulation of any one of embodiments 1-17, comprising 0.01-1% (w/w) of an antioxidant, preferably 0.05-0.8% (w/w) of an antioxidant, more preferably 0.1-0.75 (w/w) of an antioxidant, more preferably 0.2-0.7 (w/w) of an antioxidant, more preferably 0.3-0.6 (w/w) of an antioxidant, more preferably 0.4-0.5 (w/w) of an antioxidant, and most preferably about 0.5% (w/w) of an antioxidant.
19. The formulation of any one of embodiments 1-18, comprising 30-38% (w/w) of a filler, preferably 32-38% (w/w), more preferably 34-36% (w/w); in particular wherein the filler is talc.
20. The formulation of any one of embodiments 1-18, comprising 73.5% (w/w) of a first polyethylene oxide having a weight average molecular weight of M_{w} 100,000, 20% (w/w) of a second polyethylene oxide having a weight average molecular weight of M_{w} 200,000, 5% (w/w) talc, 1% (w/w) colorant and/or opacifier, and 0.5% (w/w) of an antioxidant.
21. A method of producing a pharmaceutical carrier, comprising the steps of (a) melting a formulation according to any one of embodiments 1-20, and (b) injecting the melt into a mold.
22. The method of embodiment 21, further comprising the step (c) cooling the injected melt and optionally ejecting the molded material.
23. The method of embodiment 21 or embodiment 22, wherein the pharmaceutical carrier (20) is a capsule, and at least one lid part (22) and at least one bottom part (24) is formed.
24. The method of embodiment 23, wherein at least one of the lid part (22) and the bottom part (24) has a first wall section (26, 30) with a thickness of 180-250 μm, preferably 185-225 μm, and even more preferably 190-220 μm, and a second wall section (28, 32) with a thickness of 350-450 μm, preferably 375-425 μm, more preferably 390-410 pm, and most preferably about 400 μm.
25. The method of embodiment 23, wherein the first wall section (26) of the lid part (22) defines an entire top portion of the lid part (22) and/or wherein the first wall section (30) of the bottom part (24) defines an entire bottom portion of the bottom part (24).
26. The method of any one of embodiments 23-25, wherein the pharmaceutical carrier (20) is designed such that a ratio of a lateral extension of the lid and bottom part (22, 24) to a height of the assembled lid and bottom parts (22, 24) is >1, preferably ≥1.4, more preferably ≥1.5, even more preferably ≥2, most preferably ≥2.4 and in particular ≥2.5.
27. The method of anyone of embodiments 23-26, wherein the lid part (22) and the bottom part (24) are connected to each other by a complementary closing mechanism (34); in particular wherein the closing mechanism (34) comprises a first snap part (36) which projects from the second wall section (32) of the bottom part (24) so as to face and to interact with a second snap part (38) which projects from the second wall section (28) of the lid part (22);
   more particularly wherein the first snap part (36) comprises a projection (37) adapted to engage with a corresponding projection (39) provided on the second snap part (38) so as to counteract separation of the first snap part (36) and the second snap part (38) and thus separation of the lid part (22) and the bottom part (24);
   even more particularly wherein the projection (37) provided on the first snap part (36) tapers in a direction of a free end of the first snap part (36) so as to form a first inclined engagement surface (45) adapted to engage with a second inclined engagement surface (47) formed on the projection (39) provided on the second snap part (38) which tapers in a direction of a free end of the second snap part (36);
   most preferably wherein one of the first and the second snap part (36, 38) projects from the second wall section (28, 32) of the lid part (22) or the bottom part (24) in the region of an inner circumference of the second wall section (28, 32), and wherein the other one of the first and the second snap part (36, 38) projects from the second wall section (28, 32) of the lid part (22) or the bottom part (24) in the region of an outer circumference of the second wall section (28, 32).
28. The method of embodiment 27, wherein the closing mechanism (34) further comprises an inner rib (40) which projects from the second wall section (28) of the lid part (22) or the bottom part (24) in the region of an inner circumference of the second wall section (28, 32) at a distance from the first or the second snap part (36, 38) which projects from the second wall section (28, 32) of the lid part (22) or the bottom part (24) in the region of an outer circumference of the second wall section (28, 32);
   in particular wherein the inner rib (40) tapers in a direction of a free end of the inner rib (40) so as to form a third inclined engagement surface (49) facing the first or the second snap part (36, 38) which projects from the second wall section (28, 32) of the lid part (22) or the bottom part (24) in the region of an outer circumference of the second wall section (28, 32).
29. The method of any one of embodiments 23-28, wherein the bottom part (24) is provided with an angled balcony (50) which is formed in the region of an outer surface of the second wall section (32) of the bottom part (24), in particular adjacent to the first snap part (36), and which is inclined radially outwards, in particular from an outer circumference of the first snap part (36) towards an outer surface of second wall section (32).
30. The method of any one of embodiments 23-29, wherein the pharmaceutical carrier (20) is filled with neat API.
31. The method of embodiment 23, wherein at least one of the lid part (22) and the bottom part (24) has a first wall section (26, 30) with a thickness of 180-250 μm, preferably 185-225 μm, and even more preferably 190-220 μm, and a second wall section (28, 32) with a thickness of 350-450 μm, preferably 375-425 μm, more preferably 390-410 μm, and most preferably about 400 μm, wherein the first wall section (26) of the lid part (22) defines an entire top portion of the lid part (22) and/or wherein the first wall section (30) of the bottom part (24) defines an entire bottom portion of the bottom part (24), and wherein the pharmaceutical carrier (20) is designed such that a ratio of a lateral extension of the lid and bottom part (22, 24) to a height of the assembled lid and bottom parts (22, 24) is >1, preferably ≥1.4, more preferably ≥1.5, even more preferably ≥2, most preferably ≥2.4 and in particular ≥2.5
32. The method of any one of embodiments 21-31, further comprising the step (d) sorting the carrier parts by mold cavity.
33. A pharmaceutical carrier produced by the method of any one of embodiments 21-32 using the formulation of any one of embodiments 1-20, comprising
   a lid part (22) and
   a bottom part (24),
   wherein at least one of the lid part (22) and the bottom part (24) has a first wall section (26, 30) with a thickness of 180-250 μm, preferably 185-225 μm, and even more preferably 190-220 μm, and a second wall section (28, 32) with a thickness of 350-450 μm, preferably 375-425 μm, more preferably 390-410 μm, and most preferably about 400 μm.
34. The pharmaceutical carrier of embodiment 33, wherein the pharmaceutical carrier (20) is designed such that a ratio of a lateral extension of the lid and bottom part (22, 24) to a height of the assembled lid and bottom parts (22, 24) is >1, preferably ≥1.4, more preferably ≥1.5, even more preferably ≥2, most preferably ≥2.4 and in particular ≥2.5.
35. The pharmaceutical carrier of embodiment 33 or 34, wherein a first wall section (26) of the lid part (22) defines at least a portion of a top portion of the lid part (22), in particular an entire top portion of the lid part (22).
36. The pharmaceutical carrier of any one of embodiments 33-35, wherein a second wall section (28) of the lid part (22) defines at least a portion of a side wall portion of the lid part (22) which in particular extends from the first wall section (26) of the lid part (22), along an outer circumference thereof, in the direction of the bottom part (24).
37. The pharmaceutical carrier of any one of embodiments 33-36, wherein a first wall section (30) of the bottom part (24) defines at least a portion of a bottom portion of the bottom part (24), in particular an entire bottom portion of the bottom part (24).
38. The pharmaceutical carrier of any one of embodiments 33-37, wherein a second wall section (32) of the bottom part (in 24) defines at least a portion of a side wall portion of the bottom part (24) which in particular extends from the bottom portion of the bottom part (24), along an outer circumference thereof, in the direction of the lid part (22).
39. The pharmaceutical carrier of any one of embodiment 33-38, wherein the lid part (22) and the bottom part (24) are connected to each other by a complementary closing mechanism (34).
40. The pharmaceutical carrier of embodiment 39, wherein the closing mechanism (34) comprises a first snap part (36) which projects from the second wall section (32) of the bottom part (24) so as to face and to interact with a second snap part (38) which projects from the second wall section (28) of the lid part (22).
41. The pharmaceutical carrier of embodiment 40, wherein the first snap part (36) comprises a projection (37) adapted to engage with a corresponding projection (39) provided on the second snap part (38) so as to counteract separation of the first snap part (36) and the second snap part (38) and thus separation of the lid part (22) and the bottom part (24).
42. The pharmaceutical carrier of embodiment 41, wherein the projection (37) provided on the first snap part (36) tapers in a direction of a free end of the first snap part (36) so as to form a first inclined engagement surface (45) adapted to engage with a second inclined engagement surface (47) formed on the projection (39) provided on the second snap part (38) which tapers in a direction of a free end of the second snap part (38).
43. The pharmaceutical carrier of any one of embodiments 40 to 42, wherein one of the first and the second snap part (36, 38) projects from the second wall section (28, 32) of the lid part (22) or the bottom part (24) in the region of an inner circumference of the second wall section (28, 32), and wherein the other one of the first and the second snap part (36, 38) projects from the second wall section (28, 32) of the lid part (22) or the bottom part (24) in the region of an outer circumference of the second wall section (28, 32).
44. The pharmaceutical carrier of any one of embodiments 40 to 43, wherein the closing mechanism (34) further comprises an inner rib (40) which projects from the second wall section (28, 32) of the lid part (22) or the bottom part (24) in the region of an inner circumference of the second wall section (28) at a distance from the first or the second snap part (36, 38) which projects from the second wall section (28, 32) of the lid part (22) or the bottom part (24) in the region of an outer circumference of the second wall section (28, 32).
45. The pharmaceutical carrier of embodiment 44, wherein the inner rib (40) tapers in a direction of a free end of the inner rib (40) so as to form a third inclined engagement surface (49) facing the first or the second snap part (36, 38) which projects from the second wall section (28, 32) of the lid part (22) or the bottom part (24) in the region of an outer circumference of the second wall section (28, 32).
46. The pharmaceutical carrier of any one of embodiments 33 to 45, wherein the first wall section (26) of the lid part (22), in particular in a region which is defined by a material injection point into a mold upon manufacturing of the lid part (22), is provided with a depression (42) which has a wall thickness that is larger than the wall thickness of the remaining part of the first wall section (26), but smaller than the wall thickness of the second wall section (28) of the lid part (22), a sign which indicates a cavity in which the lid part (22) was molded on a multicavity molding tool in particular being imprinted onto an inner surface of the depression (42), and/or
   wherein the first wall section (30) of the bottom part (24), in particular in a region which is defined by a material injection point into a mold upon manufacturing of the bottom part (24), is provided with a depression (44) which has a wall thickness that is larger than the wall thickness of the remaining part of the first wall section (30), but smaller than the wall thickness of the second wall section (32) of the bottom part (24), a sign which indicates a cavity in which the bottom part (24) was molded on a multicavity molding tool being imprinted onto an inner surface of the depression (44).
47. The pharmaceutical carrier of any one of embodiments 33 to 46, wherein at least one of the lid part (22) and the bottom part (24), in the region of an inner surface thereof, is provided with a plurality of inner protrusions (46) which project radially inwards from an inner surface of the second wall section (28, 32) and/or an inner surface of the inner rib (40), each of the inner protrusions (46) in particular comprising a projecting nose (48) which projects beyond the second wall section (28, 32) and/or the inner rib (40).
48. The pharmaceutical carrier of any one of embodiments 33-47, wherein the bottom part (24) is provided with an angled balcony (50) which is formed in the region of an outer surface of the second wall section (32) of the bottom part (24), in particular adjacent to the first snap part (36), and which is inclined radially outwards, in particular from an outer circumference of the first snap part (36) towards an outer surface of second wall section (32).
49. The pharmaceutical carrier of any one of embodiments 33-48, exhibiting an absolute standard mass deviation of the respective carrier parts of less than 1 mg, preferably less than 0.8 mg, more preferably less than 0.6 mg, even more preferably less than 0.4 mg, still more preferably less than 0.3 mg, still even more preferably less than 0.2 mg, and most preferably less than 0.1 mg.
50. The pharmaceutical carrier of any one of embodiments 33-49, wherein the pharmaceutical carrier exhibits a dissolution rate of at least 30%, preferably at least 40%, more preferably at least 50%, more preferably at least 60%, more preferably at least 65%, and most preferably at least 70% drug substance within 5 minutes; when tested using the ‘assay for immediate release’ described in the US Pharmacopeia 2011, section <711> using the USP apparatus II (paddle) and Fasted state simulating gastric fluid (FasSGF) at 37° C. and 50 rpm with n=3 capsules, and propanolol.HCl as the test substance.
51. The pharmaceutical carrier of any one of embodiments 33-50, wherein the pharmaceutical carrier is filled with an active pharmaceutical ingredient (API) comprising at most 5% (w/w) of an additive, preferably at most 4% (w/w), more preferably at most 3% (w/w), even more preferably at most 2% (w/w), and most preferably at most 1% (w/w).
52. A pharmaceutical carrier produced by the method of any one of embodiments 21-32 using the formulation of any one of embodiments 1-20, comprising
   a lid part (22) and
   a bottom part (24), wherein at least one of the lid part (22) and the bottom part (24) has a first wall section (26, 30) with a thickness of 180-250 μm, preferably 185-225 μm, and even more preferably 190-220 μm, and a second wall section (28, 32) with a thickness of 350-450 μm, preferably 375-425 μm, more preferably 390-410 μm, and most preferably about 400 μm, wherein the first wall section (26) of the lid part (22) defines an entire top portion of the lid part (22) and/or wherein the first wall section (30) of the bottom part (24) defines an entire bottom portion of the bottom part (24), and wherein the pharmaceutical carrier (20) is designed such that a ratio of a lateral extension of the lid and bottom part (22, 24) to a height of the assembled lid and bottom parts (22, 24) is >1, preferably ≥1.4, more preferably ≥1.5, even more preferably ≥2, most preferably ≥2.4 and in particular ≥2.5.

In the following, the present invention as defined in the embodiments is further illustrated by the following examples, which are not intended to limit the scope of the present invention. All references cited herein are explicitly incorporated by reference.

### EXAMPLES

### Example 1

In order to identify a formulation suitable for injection molding of a pharmaceutical carrier, numerous pharmaceutical polymers together with excipients which were added to impart certain functionalities have been screened (see below table).

| Functional | | Function in | |
|---|---|---|---|
| category | Examples | the carrier | Notes |
| Matrix | polyethylene oxide, kollicoat IR, | Capsule shell | In itself or in combination |
| polymer | copovidone, hydroxy propyl | matrix former | with other carrier shell |
| | cellulose, hydroxypropyl methyl | | components, must be |
| | cellulose (HPMC), | | processable via hot melt |
| | Eudragit ® copolymers (Types | | extrusion and injection |
| | E, L, N), polyvinyl pyrrolidone, | | molding, provide |
| | polyvinyl acetate, polyvinyl | | adequate mechanical |
| | acetate phthalate, pullulan, | | strength to the capsule |
| | ethyl cellulose, starch, shellac, | | shell and afford |
| | hydroxy ethyl cellulose, methyl | | appropriate drug release |
| | cellulose, HPMC acetate | | |
| | succinate, Carbopol ® | | |
| Plasticizer | polyethylene glycol (MW: 400- | Processing | Optional, to improve |
| | 8000), glycerol, xylitol, sorbitol, | aid | thermal processability of |
| | isomalt, vitamin E TPGS, | | the carrier material |
| | propylene glycol | | formulation during hot melt |
| | | | extrusion and injection |
| | | | molding |
| Solubilizer, | tartaric acid, citric acid, fumaric | Dissolution | Optional, to improve |
| Acid | acid, maleic acid, malic acid | aid (acidifying | solubility of poorly soluble, |
| | | agent) | weakly basic API (BCS |
| | | | Class II/IV) |
| Solubilizer, | tromethamine, sodium | Dissolution | Optional, improving |
| Base | bicarbonate, sodium carbonate | aid (basifying | solubility of poorly soluble, |
| | | agent) | weakly acidic API (BCS |
| | | | Class II/IV) |
| Surfactant | sodium lauryl sulfate, | Wetting | Optional, to improve |
| | poloxamers, docusate sodium, | and/or | wettability and/or solubility |
| | polyoxyethylene sorbitan fatty | solubilizing | of poorly soluble APIs |
| | acid esters, polyoxyglycerides, | agent | (BCS Class II/IV) |
| | polyoxyl hydrogenated castor | | |
| | oil | | |
| Filler | talc, mannitol, microcrystalline | Bulking agent | Optional, to improve |
| | cellulose, dicalcium phosphate, | | mechanical stiffness |
| | calcium carbonate, magnesium | | and/or thermal conductivity |
| | aluminum metasilicate | | of carrier formulation |
| Glidant | Silicon dioxide (colloidal) | Processing | Optional, improves flow of |
| | | aid | blend before injection |
| | | | molding |
| Lubricant | stearic acid, magnesium | Processing | Optional, improve release |
| | stearate, calcium stearate, | aid | from the mold after |
| | hydrogenated vegetable oil, | | injection molding |
| | sodium stearyl fumarate (SSF), | | |
| | stearyl alcohol, glyceryl | | |
| | behenate | | |
| Anti-tackifier | talc | Processing | Reduce sticking behavior |
| | | and texture | of capsule shell |
| | | aid | |
| Disintegrant | crospovidone, sodium starch | Dissolution | Optional, improve |
| | glycolate, sodium | aid | disintegration behavior of |
| | croscarmellose | | capsule shells |
| Opacifiers | titanium dioxide, iron oxide, | Identification, | Optional, improve |
| and | lake pigments, mica-based | cosmetic, | aesthetics of capsule shell |
| colorants | pigments (Candurin), Opadry ® | protective | |
| | (formulated pigments) | | |
| Flavors and | sucrose, mannitol, sorbitol, | Taste | Optional, improve |
| sweeteners | saccharin, isomalt, xylitol | masking | palatability of the capsule |
| | | | shell material |
| Antioxidant | butylated hydroxy toluene, | Stability | Optional, prevent oxidation |
| | butylated hydroxy anisole, | | of matrix polymer during |
| | vitamin E, vitamin E TPGS, | | storage |
| | ascorbic acid, isoascorbic acid, | | |
| | citric acid, propyl gallate | | |

Specifically, in order to examine the flow properties of different polymers, test plates with a geometry of 25 mm×25 mm+‘variable thickness’ have been prepared by injection moulding. Parameter of interest was in particular the flow length. The following is an excerpt of results obtained for different materials examined at a plate thickness of 0.2 mm.

For example, Eudragit E100 granules (0.2 mm) were tested at different temperatures of the bulk mass and of the tool. The latter one was adjusted either by pressure or by distance.

| | Flow length [mm] | | | | |
|---|---|---|---|---|---|
| | Tool temperature [° C.] | | | | |
| | By pressure | | By distance | | |
| Bulk mass temperature [° C.] | 40 | 50 | 20 | 40 | 50 |
| 130 | 5 | 5.5 | | | |
| 140 | 6 | 6 | | | 7 |
| 150 | 7 | 5.5 | | 9 | |
| 160 | | | 5.5 | | |
| 170 | | | 4.5 | | |
| 180 | | | 4.5 | | |

It was found that starting from 170° C. bulk mass temperature, the mass starts foaming, leading to dosing problems and deformation of the test plates. At 140° C. the filling is rather bad. At 50° C. tool temperature, the mold is too elastic and sticky. Best results for Eudragit E100 were obtained at a bulk mass temperature of 150° C. and a tool temperature of 40° C. (9 mm).

In another line of experiments, Soluplus powder was examined in the same way than Eudragit E100:

| | Flow length [mm] | | |
|---|---|---|---|
| | Tool temperature [° C.] | | |
| Bulk mass temperature [° C.] | 30 | 45 | 60 |
| 140 | 4 | 4 | 5 |
| 150 | 6.5 | 6.5 | 8, sticks |
| 160 | 9.5 | sticks | sticks |
| 170 | foam | foam | foam |

Isomalt (GalenIQ 990) shows a too low viscosity at the tested temperatures, and only resulted in a brittle, porous mass.

Kollicoat IR showed unsatisfying material flow at 160-170° C., and very low flow lengths at 180-190° C., irrespective of the mould temperature. The best result was obtained at a bulk mas temperature of 200° C. and a mould temperature of 80° C. (4.5 mm). Above 210° C. the material becomes turbide and the plates start wrapping after mould release.

Klucel EF and ELF were shown to be difficult in processing. Starting at 180-190° C. of the bulk mass temperature, the material begins foaming and turning brownish, while 130° C. is too cold. At a mould temperature of 45° C., the gate keeps in the mould. Best result for both materials was observed at 140° C. bulk mass temperature/30° C. tool temperature, showing a flow length of 10 mm.

Copovidone was found to be very brittle, causing problems especially during the opening of the mold. Even plates with a thickness of 0.3 mm could not be formed.

Polyox N10 showed a cavity filling of approximately 80%, showing a maximum flow length of 24 mm at 150° C. bulk mass temperature and 30° C. mould temperature. The material was found very workable.

Hence, at a thickness of 0.2 mm, the results can be summarized as follows:

| Polymer | approx. flow length [mm] |
|---|---|
| Polyox | 24 |
| Klucel ELF | 10 |
| Klucel EF | 10 |
| Soluplus powder | 9.5 |
| Eudragit E100 | 9 |
| Kollicoat IR | 4.5 |
| Isomalt (GalenIQ 990) | — |
| Copovidone | — |

The screening was repeated at a plate thickness of 0.1 mm, which represents the intended wall thickness of the pharmaceutical carrier to be prepared by injection molding. Polyox was the only tested polymer being capable of realising a wall thickness of 0.1 mm (flow length 4.5 mm).

These foregoing screening studies showed that polyethylene oxide (PEO) was the polymer which had the most suitable properties for injection molding, particularly for challenging shapes and was selected as the primary shell ingredient. Ratios of different molecular weight PEO were tested, and it was found that PEO having a weight average molecular weight of 49,000 to 188,000 were found to achieve a formulation with the correct physico-chemical properties.

In a further comparative assay, it was found that the processibility of the PEO could be further improved in terms of faster cycle times, lower pressure and improved ejection and melt flow when adding 3-7% by weight of talc as an anti-tackifier. In the following experiment, a formulation similar to formulation 11 described below without an anti-tackifier was compared to formulation 11 comprising 5% talc as an antitackifier:

| Talc | | − | + |
|---|---|---|---|
| Mold temperature | ° C. | 150 | 150 |
| Ejector plate temperature | ° C. | 30 | 30 |
| Cycle time, Top | S | 34 | 18 |
| Cycle time, Bottom | S | 34 | 18 |
| Injection pressure, Top | Bar | 2000 | 1200 |
| Injection pressure, Bottom | Bar | 2000 | 1400 |
| Melt flow, Top | Rank order (1-4) | 3 | 4 |
| Melt flow, Bottom | Rank order (1-4) | 3 | 4 |
| Ease of ejection, Top | Rank order (1-4) | 3 | 4 |
| Ease of ejection, Bottom | Rank order (1-4) | 3 | 4 |
| Ejector mark, Top | Rank order (1-4) | 4 | 4 |
| Ejector mark, Bottom | Rank order (1-4) | 3 | 3 |

The following formulations were found to be particularly suitable for injection molding of a pharmaceutical carrier:

| | Formula 1 | |
|---|---|---|
| Component | (MW 94,000) | Function |
| Polyethylene oxide, | 94.00% | Matrix former |
| MW: 100000 | | |
| [PolyOx N10] | | |
| Talc | 5.00% | Anti-tackifier & |
| | | thermal conductivity |
| | | enhancer |
| Excipient(s) | 1.00% | |

| | Formula 2 | |
|---|---|---|
| Component | (MW 188,000) | Function |
| Polyethylene oxide, | 94.00% | Matrix former |
| MW: 200000 | | |
| [PolyOx N80] | | |
| Talc | 5.00% | Anti-tackifier & |
| | | thermal conductivity |
| | | enhancer |
| Excipient(s) | 1.00% | |

| | Formula 3 | |
|---|---|---|
| Component | (MW 172,000) | Function |
| Polyethylene oxide, | 55.00% | Primary matrix former |
| MW: 100000 | | |
| [PolyOx N10] | | |
| Polyethylene oxide, | 39.00% | Secondary matrix former |
| MW: 300000 | | |
| [PolyOx N750] | | |
| Talc | 5.00% | Anti-tackifier & |
| | | thermal conductivity |
| | | enhancer |
| Excipient(s) | 1.00% | |

| | Formula 4 | |
|---|---|---|
| Component | (MW 102,000) | Function |
| Polyethylene oxide, | 90.00% | Primary matrix former |
| MW: 100000 | | |
| [PolyOx N10] | | |
| Polyethylene oxide, | 4.00% | Secondary matrix former |
| MW: 300000 | | |
| [PolyOx N750] | | |
| Talc | 5.00% | Anti-tackifier & |
| | | thermal conductivity |
| | | enhancer |
| Excipient(s) | 1.00% | |

| | Formula 5 | |
|---|---|---|
| Component | (MW 182,720) | Function |
| Polyethylene oxide, | 60.00% | Primary matrix former |
| MW: 300000 | | |
| [PolyOx N750] | | |
| Polyethylene oxide, | 34.00% | Secondary matrix former |
| MW: 8000 | | |
| [PEG8000] | | |
| Talc | 5.00% | Anti-tackifier & |
| | | thermal conductivity |
| | | enhancer |
| Excipient(s) | 1.00% | |

| | Formula 6 | |
|---|---|---|
| Component | (MW 95,120) | Function |
| Polyethylene oxide, | 30.00% | Primary matrix former |
| MW: 300000 | | |
| [PolyOx N750] | | |
| Polyethylene oxide, | 64.00% | Secondary matrix former |
| MW: 8000 | | |
| [PEG8000] | | |
| Talc | 5.00% | Anti-tackifier & |
| | | thermal conductivity |
| | | enhancer |
| Excipient(s) | 1.00% | |

| | Formula 7 | |
|---|---|---|
| Component | (MW 164,000) | Function |
| Polyethylene oxide, | 80.00% | Primary matrix former |
| MW: 100000 | | |
| [PolyOx N10] | | |
| Polyethylene oxide, | 14.00% | Secondary matrix former |
| MW: 600000 | | |
| [PolyOx 205] | | |
| Talc | 5.00% | Anti-tackifier & |
| | | thermal conductivity |
| | | enhancer |
| Excipient(s) | 1.00% | |

| | Formula 8 | |
|---|---|---|
| Component | (MW 114,000) | Function |
| Polyethylene oxide, | 90.00% | Primary matrix former |
| MW: 100000 | | |
| [PolyOx N10] | | |
| Polyethylene oxide, | 4.00% | Secondary matrix former |
| MW: 600000 | | |
| [PolyOx 205] | | |
| Talc | 5.00% | Anti-tackifier & |
| | | thermal conductivity |
| | | enhancer |
| Excipient(s) | 1.00% | |

| | Formula 9 | |
|---|---|---|
| Component | (MW 185,120) | Function |
| Polyethylene oxide, | 30.00% | Primary matrix former |
| MW: 600000 | | |
| [PolyOx 205] | | |
| Polyethylene oxide, | 64.00% | Secondary matrix former |
| MW: 8000 | | |
| [PEG8000] | | |
| Talc | 5.00% | Anti-tackifier & |
| | | thermal conductivity |
| | | enhancer |
| Excipient(s) | 1.00% | |

| | Formula 10 | |
|---|---|---|
| Component | (MW 125,920) | Function |
| Polyethylene oxide, | 20.00% | Primary matrix former |
| MW: 600000 | | |
| [PolyOx 205] | | |
| Polyethylene oxide, | 74.00% | Secondary matrix former |
| MW: 8000 | | |
| [PEG8000] | | |
| Talc | 5.00% | Anti-tackifier & |
| | | thermal conductivity |
| | | enhancer |
| Excipient(s) | 1.00% | |

The following is an example of a particular balanced formulation, which is also used in the following experiments.

| Component | Formulation 11 | Function |
|---|---|---|
| Polyethylene oxide, | 73.50% | Primary matrix former |
| MW: 100000 | | |
| [PolyOx N10] | | |
| Polyethylene oxide, | 20.00% | Secondary matrix former |
| MW: 200000 | | |
| [PolyOx N80] | | |
| Talc | 5.00% | Anti-tackifier & |
| | | thermal conductivity |
| | | enhancer |
| Colorant | 1.00% | Appearance |
| Antioxidant | 0.50% | |

The following formulations are also found to be suitable for injection molding of a pharmaceutical carrier:

| Formulation | 12 | 13 | 14 | 15 | 16 | 17 | 18 | 19 |
|---|---|---|---|---|---|---|---|---|
| Average MW | 73000 | 49200 | 63000 | 51400 | 50000 | 49200 | 53000 | 66300 |
| Polyox N10 (MW 100,000) | 50 | 37.8 | 41 | 43.4 | 42 | 37.8 | 41 | 45.7 |
| Polyox N80 (MW 200,000) | 11.5 | 5.7 | 11 | 4 | 4 | 5.7 | 6 | 10.3 |
| anti-tackifier (talc) | 5 | 5 | 5 | 5 | 5 | 5 | 5 | 5 |
| antioxidant | 0.5 | 0.5 | 0.5 | 0.5 | 0.5 | 0.5 | 0.5 | 0.5 |
| pigment | 1 | 1 | 1 | 1 | 1 | 1 | 1 | 0 |
| Filler (talc) | 30 | 30 | 36.7 | 38 | 38 | 30 | 36 | 30.9 |
| Other excipients | 2 | 20 | 4.8 | 8.1 | 9.5 | 20 | 10.5 | 7.6 |

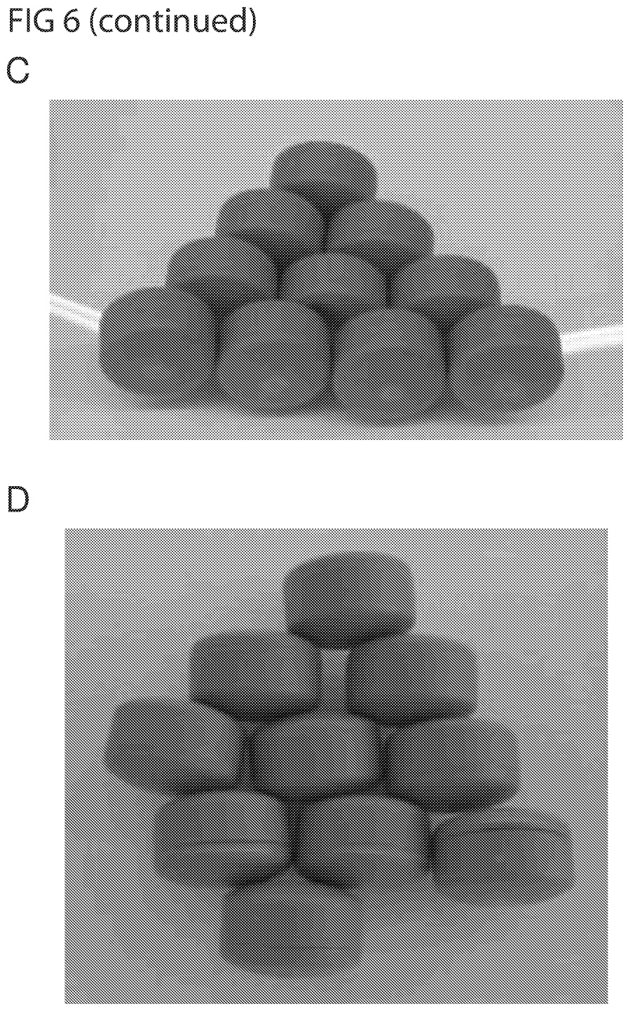

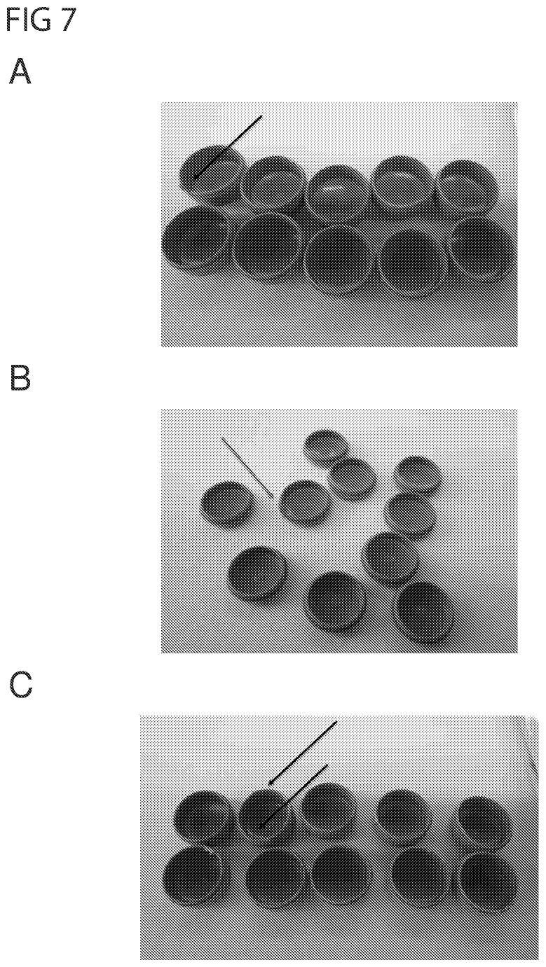

However, during further testing, it was found that pharmaceutical carriers prepared from molts having a weight average molecular weight of less than M_{W} 94,000 exhibited an undesirable integrity and/or stability. For example, the test pharmaceutical carriers were stored at (i) 25° C. and 60% room humidity, 30° C. and 75% room humidity, or 40° C. and 75% room humidity. The tested carriers did not close very well after 3 months storage (cf. FIG. 6A and 6C), and even worse after 6 months storage (cf. FIG. 6B and 6D), most likely due to deformations resulting from storage (cf. FIG. 7). Such problems did not occur when using molts having a weight average molecular weight of at least M_{W} 94,000.

Moreover, pharmaceutical carriers made up of a polyethylene oxide polymer having a weight average molecular weight of more than M_{W} 188,000 will result in pharmaceutical carriers in which the dissolution rate is unsatisfying. The relationship between an increase of the molecular weight polymer and a decrease in dissolution rate has been studied previously (see for example, Ueberreiter K. The solution process. In: Crank J, Park G S, editors. Diffusion in polymers. New York, N.Y.: Academic Press; 1968. p. 219-57; Miller-Chou, B and Koenig, J., A review of polymer dissolution, Prog. Polym. Sci. 2003, 28: 1223-1270).

### Example 2

Dissolution of a model compound, Propranolol.HCL, directly filled with no added excipients in capsules of formulation 11 has been carried out and compared to commercially available hard gelatin and HPMC capsules. Results show that the dissolution profile from the carrier of the present disclosure is similar to hard gelatin capsules (HGC), giving rapid and total release of API (cf. FIG. 4). Importantly, there is no lag time, unlike commercially available capsules comprised of synthetic materials (c.f., FIG. 4, HPMC).

### Example 3

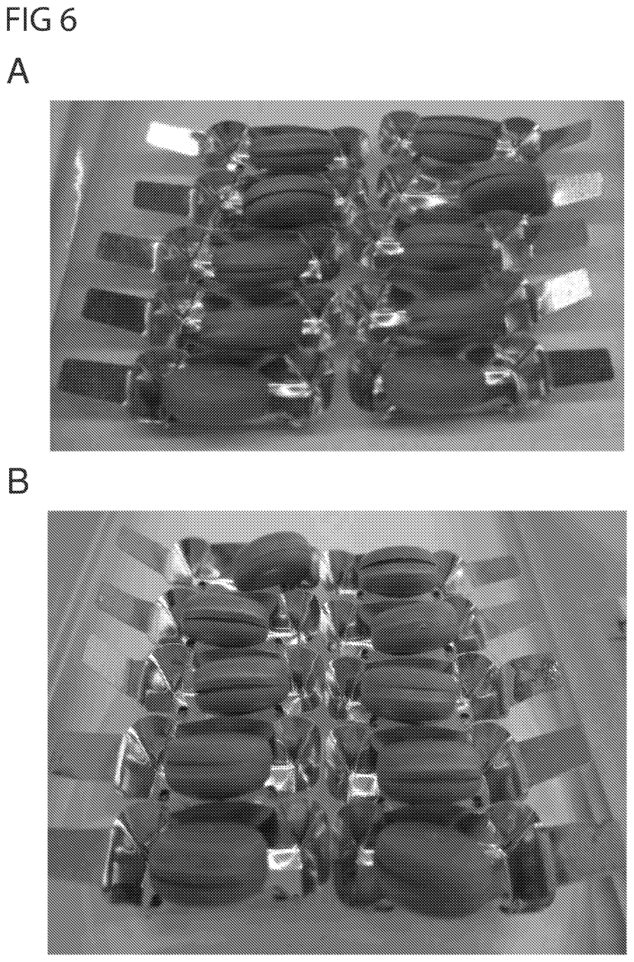

The weights of carriers (formulation 11) molded from a multi cavity tool were measured (cf. FIG. 5). The standard deviation was calculated and compared to commercially available hard gelatin capsules of a comparable fill volume (FIG. 5, left bar). Results show that even if the carriers are analyzed at random (i.e. not sorted by cavity) the standard deviations are very low, roughly third of that for a similar volume hard gelatin capsule (FIG. 5, middle bar). If the carrier parts are sorted by cavity the standard deviation in weight is extremely low, an order of magnitude lower than for the hard gelatin capsule (FIG. 5, right bar).

For products which require weight control, this can be achieve via gross weighing of filled carriers, rather than the current standard of gross-tare weighing, halving the time required for weight control.

1. A formulation for injection molding of a pharmaceutical carrier, wherein the formulation comprises 43.5-97% (w/w) of one or more polyethylene oxide polymer having a weight average molecular weight of M_{W} 94,000-188,000; and optionally one or more excipients.

2. The formulation of claim 1, wherein said polyethylene oxide polymer comprises one or more polyethylene oxide having a weight average molecular weight of about M_{W} 100,000.

3. The formulation of claim 1, wherein said polyethylene oxide polymer comprises 35-80% (w/w) of a first polyethylene oxide having a weight average molecular weight of M_{W} 100,000; and 4-28.5% (w/w) of a second polyethylene oxide having a weight average molecular weight of M_{W} 200,000.

4. The formulation of claim 3, wherein the formulation comprises 41-77.5% (w/w) of said second polyethylene oxide.

5. The formulation of claim 1, wherein the formulation comprises 3-7% (w/w) of an anti-tackifier; and wherein the anti-tackifier is talc.

6. (canceled)

7. The formulation of claim 1, wherein the excipient is at least one selected from the list consisting of colorant, antioxidant, opacifier, acid solubilizer, base solubilizer, surfactant, filler, glidant, lubricant, disintegrant, flavour, sweetener, and
wherein the acid solubilizer is selected from the group consisting of tartaric acid, citric acid, fumaric acid, maleic acid, malic acid, and any combination thereof; and/or
wherein the base solubilizer is selected from the group consisting of tromethamine, sodium bicarbonate, sodium carbonate, and any combination thereof; and/or
wherein the surfactant is selected from the group consisting of sodium lauryl sulfate, poloxamer, docusate sodium, polyoxyethylene sorbitan fatty acid esters, polyoxyglycerides, polyoxyl hydrogenated castor oil, and any combination thereof; and/or
wherein the filler is selected from the group consisting of talc, mannitol, microcrystalline cellulose, dicalcium phosphate, calcium carbonate, magnesium aluminium metasilicate, and any combination thereof; and/or
wherein the glidant is colloidal silicon dioxide, and/or
wherein the lubricant is stearic acid or one of its salts such as magnesium stearate or calcium stearate, hydrogenated vegetable oil, sodium stearyl fumarate (SSF), stearyl alcohol, glyceryl behenate, or any combination thereof; and/or
wherein the disintegrant is crospovidone, sodium starch glycolate, sodium croscarmellose, or any combination thereof; and/or
wherein the opacifier and colorant is selected from titanium dioxide, iron oxide, lake pigments, mica-based pigments, formulated pigments, and any combination thereof; and/or wherein the antioxidant is selected from the group consisting of butylated hydroxy toluene, butylated hydroxy anisole, vitamin E, vitamin E TPGS, ascorbic acid, isoascorbic acid, citric acid, propyl gallate, and any combination thereof.

8. The formulation of claim 1, comprising 0-6% (w/w) of one or more colorant and/or opacifier, and/or comprising 0.01-1% (w/w) of an antioxidant.

9. The formulation of claim 1, comprising 30-38% (w/w) of a filler; and wherein the filler is talc.

10. The formulation of claim 1, comprising 73.5% (w/w) of a first polyethylene oxide having a weight average molecular weight of M_{w} 100,000, 20% (w/w) of a second polyethylene oxide having a weight average molecular weight of M_{w} 200,000, 5% (w/w) talc, 1% (w/w) colorant and/or opacifier, and 0.5% (w/w) of an antioxidant.

11. A method of producing a pharmaceutical carrier, comprising the steps of
(a) melting a formulation according to claim 1, and
(b) injecting the melt into a mold, and
(c) optionally cooling the injected melt and optionally ejecting the molded material.

12. The method of claim 11, wherein the pharmaceutical carrier (20) is a capsule, and at least one lid part (22) and at least one bottom part (24) is formed, and wherein at least one of the lid part (22) and the bottom part (24) has a first wall section (26, 30) with a thickness of 180-250 μm, and a second wall section (28, 32) with a thickness of 350-450 μm, and wherein the first wall section (26) of the lid part (22) defines an entire top portion of the lid part (22) and/or wherein the first wall section (30) of the bottom part (24) defines an entire bottom portion of the bottom part (24).

13. (canceled)

14. The method of claim 12, wherein the lid part (22) and the bottom part (24) are connected to each other by a complementary closing mechanism (34);
wherein the closing mechanism (34) comprises a first snap part (36) which projects from the second wall section (32) of the bottom part (24) so as to face and to interact with a second snap part (38) which projects from the second wall section (28) of the lid part (22);
wherein the first snap part (36) comprises a projection (37) adapted to engage with a corresponding projection (39) provided on the second snap part (38) so as to counteract separation of the first snap part (36) and the second snap part (38) and thus separation of the lid part (22) and the bottom part (24);
wherein the projection (37) provided on the first snap part (36) tapers in a direction of a free end of the first snap part (36) so as to form a first inclined engagement surface (45) adapted to engage with a second inclined engagement surface (47) formed on the projection (39) provided on the second snap part (38) which tapers in a direction of a free end of the second snap part (36).

15. The method of claim 11, wherein the pharmaceutical carrier (20) is designed such that a ratio of a lateral extension of the lid and bottom part (22, 24) to a height of the assembled lid and bottom parts (22, 24) is >1.

16. The method of claim 11, further comprising the step (d) sorting the carrier parts by mold cavity.

17. A pharmaceutical carrier produced by the method of claim 11 using the formulation for injection molding of a pharmaceutical carrier, wherein the formulation comprises 43.5-97% (w/w) of one or more polyethylene oxide polymer having a weight average molecular weight of M_{W} 94,000-188,000; and optionally one or more excipients, comprising
a lid part (22) and
a bottom part (24),
wherein each of the lid part (22) and the bottom part (24) has a first wall section (26, 30) with a thickness of 180-250 μm and a second wall section (28, 32) with a thickness of 350-450 μm, and wherein the first wall section (26) of the lid part (22) defines an entire top portion of the lid part (22) and/or wherein the first wall section (30) of the bottom part (24) defines an entire bottom portion of the bottom part (24).

18. The pharmaceutical carrier of claim 17, exhibiting a standard mass deviation of the respective carrier parts of less than 1 mg.

19. The pharmaceutical carrier of claim 17, wherein the pharmaceutical carrier exhibits a dissolution rate of at least 30% drug substance within 5 minutes; when tested using the ‘assay for immediate release’ described in the US Pharmacopeia 2011, section <711> using the USP apparatus II (paddle) and Fasted state simulating gastric fluid (FasSGF) at 37° C. and 50 rpm with n=3 capsules, and propanolol.HCl as the test substance.

20. The pharmaceutical carrier of claim 17, wherein the pharmaceutical carrier is filled with a neat active pharmaceutical ingredient (API) comprising at most 5% (w/w) of an additive.
